(19) United States
(12) Patent Application Publication
(10) Pub. No.: US 2025/0011865 A1
(43) Pub. Date: Jan. 9, 2025

(54) KIT FOR CALIBRATION OF ISOTHERMAL POLYMERASE CHAIN REACTION (PCR) ANALYZER AND USE THEREOF

(71) Applicant: National Institute of Metrology, China, Beijing (CN)

(72) Inventors: Yunhua GAO, Beijing (CN); Xian CHEN, Beijing (CN); Di WANG, Beijing (CN); Yue FEI, Beijing (CN); Zhidong WANG, Beijing (CN); Xiao WU, Beijing (CN); Song LU, Beijing (CN)

(21) Appl. No.: 18/600,305

(22) Filed: Mar. 8, 2024

(30) Foreign Application Priority Data
Jul. 5, 2023 (CN) 202310817917.X

(51) Int. Cl. C12Q 1/6876; C12N 15/63; C12Q 1/48; C12Q 1/6844

(52) U.S. Cl. CPC C12Q 1/6876; C12N 15/63; C12Q 1/485; C12Q 1/6844; C12Q 2600/166; G01N 2333/9126

(57) ABSTRACT

Provided is a kit for calibration of an isothermal polymerase chain reaction (PCR) analyzer and use thereof. The kit includes a standard substance, an amplification primer set, a reaction buffer, a polymerase, a dye, and a negative control. The standard substance is a DNA plasmid with a gradient concentration of 10⁰ copies/μL to 10⁶ copies/μL; the amplification primer set has nucleotide sequences shown in SEQ ID NO: 1 to SEQ ID NO: 6; the polymerase is a Bst DNA polymerase; and the dye is a loop-mediated isothermal amplification (LAMP) fluorescent dye (with excitation: 485 nm, emission: 498 nm, and a detection channel of SYBR® Green I or a FAM channel). The kit and a technology for calibration of an isothermal PCR instrument meet calibration demands of the isothermal PCR instrument and fill a technical gap.

### CROSS-REFERENCE TO RELATED APPLICATION

This patent application claims the benefit and priority of Chinese Patent Application No. 202310817917.X filed with the China National Intellectual Property Administration on Jul. 5, 2023, the disclosure of which is incorporated by reference herein in its entirety as part of the present application.

### SEQUENCE LISTING

The instant application contains a Sequence Listing which has been submitted electronically in XML format and is hereby incorporated by reference in its entirety. Said XML copy, created on Mar. 8, 2024, is named Sequence Listing BHIW-005.xml and is 21 kilobytes in size.

### TECHNICAL FIELD

The present disclosure belongs to the technical field of genetic engineering, and in particular relates to a kit for calibration of an isothermal polymerase chain reaction (PCR) analyzer and use thereof.

### BACKGROUND

Polymerase chain reaction (PCR), as a molecular biology technique for amplifying specific DNA fragments, can complete amplification in denaturation, annealing, and extension steps using primers and thermostable enzymes. Compared with traditional PCR instruments, isothermal PCR instruments are thermal cyclers that do not rely on accurate temperature control to conduct DNA amplification at a constant temperature with high reaction efficiency and reaction rate, thus greatly meeting the personalized needs for nucleic acid testing in society. As a result, different types of isothermal PCR instruments continue to emerge and are gradually introduced to the market. The isothermal PCR instrument greatly lowers the threshold for nucleic acid detection, allowing the nucleic acid detection to be separated from specialized PCR laboratories and then applied to more testing scenarios. Accordingly, the research on calibration technology for isothermal PCR instruments that are automated and simplified as well as have different application scenarios can meet a great demand for isothermal PCR instrument calibration in the society. This effectively guarantees accurate and effective technical support for the detection results of COVID-19, African swine fever, avian influenza and other epidemics based on isothermal PCR detection, exhibiting significant social and economic benefits.

Although the demand for calibration of isothermal PCR instruments is increasing rapidly, the lack of calibration technology and standard kits that match the isothermal PCR instruments, as well as the lack of calibration procedures, have seriously hindered the development of calibration work. At present, the calibration methods and calibration kits of real-time fluorescence quantitative PCR instruments cannot be used for the calibration of isothermal PCR instruments, which is mainly due to the following problems: (1) the primer-probe system is not suitable, and the calibration of isothermal PCR instruments requires special design of the system; (2) the amplification enzyme reaction system is not suitable, and a special enzyme reaction system suitable for isothermal amplification is required; and (3) there are different calibration items with different temperatures, but the isothermal PCR is conducted at a constant temperature without a temperature-changing process.

The calibration for an isothermal PCR instrument consists of two aspects. One is to calibrate a temperature field performance of the isothermal PCR instrument, and involves temperature accuracy, temperature uniformity and other indicators. The other is to calibrate optical performance, repeatability, sensitivity and other parameters of the equipment using calibration reagents to comprehensively evaluate an equipment performance.

The isothermal PCR instrument performs DNA amplification at a constant temperature without changing the temperature, shows high reaction efficiency and reaction rate, and is suitable for application scenarios such as on-site detection and point-of-care diagnosis. Currently, common isothermal nucleic acid amplification technologies include: loop-mediated isothermal amplification (LAMP), nucleic acid sequence-based amplification (NASBA), rolling circle amplification (RCA), and recombinase polymerase amplification (RPA).

In the LAMP technique, 6 to 8 regions of a target DNA are recognized through 4 to 6 primers, and a reaction at about 65° C. is conducted using a DNA polymerase (Bst DNA polymerase) with displacement activity, where a loop structure formed by two of the primers triggers the amplification. The LAMP technique has high amplification efficiency and a short reaction time, and is suitable for application scenarios such as on-site detection and point-of-care diagnosis.

In the NASBA technique, amplification is guided through a pair of primers with a T7 promoter sequence, and a reaction is conducted at about 41° C. using a T7 RNA polymerase. NASBA has a strong specificity and a high sensitivity, but shows a high reaction cost (three enzymes required) and relatively complex reaction components.

In the RCA technique, isothermal amplification of a circular DNA template is achieved through strand displacement synthesis of a primer and the circular DNA template under the action of a DNA polymerase. The RCA technique has high throughput, sensitivity, and specificity, but shows a high cost in synthesis of Zigzag probes and background interference.

In the RPA technique, a primer is bound to a double-stranded DNA through a recombinase. The recombinase and a single-stranded binding protein form a D-loop recombination structure, and amplification is initiated by a strand-displacement DNA polymerase at around 37° C. The RPA technique shows rapid detection, but is difficult to avoid some non-specific amplifications.

Compared with the other three isothermal amplification technologies, LAMP technique exhibits high amplification efficiency and a short reaction time, while most of kits on the market are based on the LAMP amplification system. In view of this, it is urgent to construct a kit for calibration of isothermal PCR instruments based on the LAMP system to form a calibration method. Moreover, a LAMP-based kit for calibration of the isothermal PCR instruments can comprehensively evaluate a detection performance of the isothermal PCR instruments; meanwhile, an isothermal PCR instrument calibrated by the LAMP-based kit for calibration of the isothermal PCR instruments can normally conduct isothermal PCR reactions of other amplification systems.

So far, there is a rapidly increasing demand for the calibration of isothermal PCR instruments, but there is also a lack of calibration technology and standard kits that match the isothermal PCR instruments and the calibration procedures are missing. Therefore, it is of great significance to establish a calibration method that matches the isothermal PCR instrument and to develop a special standard kit for the isothermal PCR instrument. The kit can effectively support the measurement market supervision of isothermal PCR instrument performance, effectively ensure the normal performance parameters of isothermal PCR instrument in the market, and prop up the accuracy and reliability of nucleic acid detection results.

### SUMMARY

A first objective of the present disclosure is to provide a primer set for calibration of an isothermal PCR analyzer, where the primer set has the nucleotide sequences shown in SEQ ID NO: 1 to SEQ ID NO: 6.

A second objective of the present disclosure is to provide a kit for calibration of an isothermal PCR analyzer, including the primer set.

Preferably, the kit further includes a standard substance, a reaction buffer, a polymerase, and a dye.

More preferably, the standard substance is a DNA plasmid with a gradient concentration of 10⁰ copies/μL to 10⁶ copies/μL.

More preferably, the DNA plasmid is a pMD18 plasmid with a specific sequence shown in SEQ ID NO: 13.

SEQ ID NO: 13:
ACTCTTCCAG CCTTCCTTCC TGGGTGAGTG
GAGACTGTAATCTTGGCTCACCCTCATGAGGGTTACCCCTCGGGGCTGTG
CTGTGGAAGCTATCTCCTGCCCTCATTTCCCTCTCAGGCATGGAGTCCTG
TGGCATCCACGTTTCTACCTTCAACTCCATCATGAAGTGTCTGGTGGACA
TCCGCAAAGACCTGTACGCAACACAGTGCTGTCTCCGCGCACCACCATCC
AAGGGGTGGCATTGCCGACAGGATGCAGAA
GGAGATCACT GCCCTGGCAC CCAGCACAAT CTTGATCTTG.

More preferably, the reaction buffer includes the following components: 20 mM of Tris-HCl, 10 mM of (NH₄)₂SO₄, 50 mM of KCl, 8 mM of MgSO₄, 0.1% of Tween 20, and 1.4 mM each of dNTPs; the polymerase is a Bst DNA polymerase, and the dye is a LAMP fluorescent dye; and a detection channel corresponding to the LAMP fluorescent dye is SYBR® Green I or a FAM channel.

More preferably, the kit further includes a negative control.

A third objective of the present disclosure is to provide a LAMP reaction system for calibration of an isothermal PCR instrument, where the LAMP reaction system has a volume of 25 μL and includes the following components: 12.5 μL of 2× reaction buffer, 1 μL of Bst DNA polymerase, 0.5 μL of 50×LAMP dye, 5 pmol of SEQ ID NO: 1, 5 pmol of SEQ ID NO: 2, 40 pmol of SEQ ID NO: 3, 40 pmol of SEQ ID NO: 4. 10 pmol of SEQ ID NO: 5, 10 pmol of SEQ ID NO: 6, and 1 μL of standard substance, and ultrapure water added to make up to 25 μL.

A fourth objective of the present disclosure is to provide use of the primer set in calibration of an isothermal PCR analyzer.

A fifth objective of the present disclosure is to provide use of the kit in calibration of an isothermal PCR analyzer.

Compared with the prior art, the present disclosure has the following beneficial effects:

(1) In the present disclosure, a dedicated kit for calibration of an isothermal PCR instrument is provided, and is applicable to all models of isothermal PCR instruments.

(2) In the present disclosure, an exclusive calibration technology for isothermal PCR instruments is provided.

(3) In the present disclosure, the kit and technology for calibration of an isothermal PCR instrument meet the calibration requirements of isothermal PCR instruments.

### BRIEF DESCRIPTION OF THE DRAWINGS

FIG. 1 shows a schematic diagram of the kit for calibration of isothermal PCR in the present disclosure;

FIG. 2 shows amplification effects of 3 sets of base primers in a LAMP repeated experiment in Example 1;

FIG. 3 shows an amplification effect of a primer set in a LAMP repeated experiment in Example 2;

FIG. 4 shows LAMP amplification effects at different concentrations in Example 4;

FIG. 5 shows a measurement range of the LAMP reaction in Example 4; and

FIG. 6 shows a measurement range of the LAMP reaction for an isothermal PCR instrument in Example 5.

### DETAILED DESCRIPTION OF THE EMBODIMENTS

In the present disclosure, base primers and loop primers are designed based on optimization of Tm value, dG value, GC content, and distance between primers of a target sequence of the standard substance using LAMP and PrimerExplorer V5 software. The standard substance, amplification primers, amplification reagents, and water are configured into a 25 μL LAMP reaction system to allow isothermal amplification at 65° C., so as to obtain an exponential amplification inflection point time (min) within 40 min.

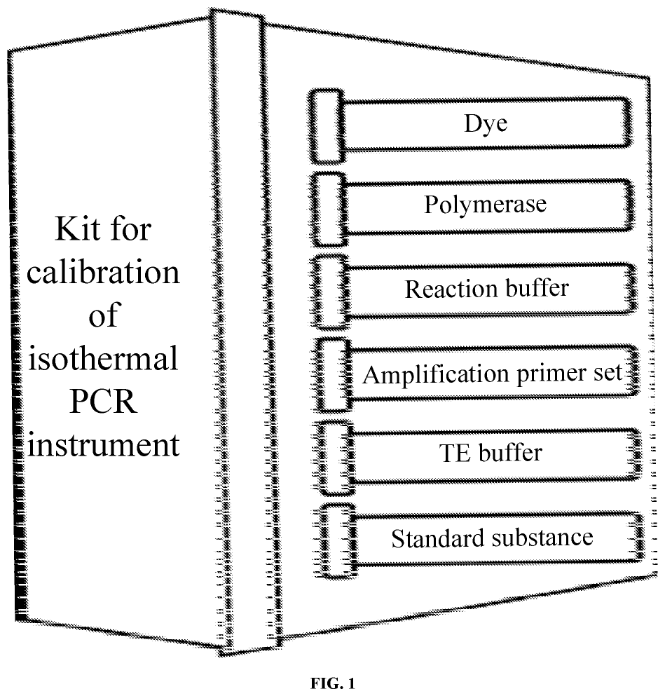

The kit for calibration of isothermal PCR (FIG. 1) includes:

TABLE 1

| No. | Component |
|---|---|
| 1 | Standard substances (S1, S2, S3, S4, S5, S6, S7) |
| 2 | Negative control (TE buffer (10 mM, pH = 7.8)) |
| 3 | Amplification primer set |
| 4 | 2 × reaction buffer |
| 5 | Bst DNA polymerase |
| 6 | 50 × LAMP dye |

(1) Standard substance:

The standard material is plasmid DNA, and its characteristic value is accurately determined using digital PCR, with a concentration of (10⁰-10⁶) copies/μL. The standard material is used as a standard in the kit for calibration of parameters such as sensitivity and repeatability of an isothermal PCR instrument.

### Amplification Primer Set:

TABLE 2

| Sequence ID | Primer | |
|---|---|---|
| No. | name | Nucleotide sequence |
| SEQ ID No: 1 | F3 | CTGGGTGAAAGGAGACTGT |
| SEQ ID No: 2 | B3 | AGACAGGGCTGTGTTGGC |
| SEQ ID No: 3 | FIP | TGAGAGGGAAATGATTTCAGGACATGAGGGTTACCCC |
| | | TCGG |
| SEQ ID NO: 4 | BIP | GGCATGGAGTCCTCACCCATCGTACAGGTCTTTGCGG |
| | | ATGT |
| SEQ ID NO: 5 | LF | TAGCTTAATCAGGAGAGCC |
| SEQ ID NO: 6 | LB | ACCTTCTTCTCCATCATGAAGTG |

### (2) Reaction System (25 μL):

TABLE 3

| | | |
|---|---|---|
| Component | Working concentration | Volume (μL) |

| | | |
|---|---|---|
| Reaction buffer | 1× | 12.5 |

| | | | |
|---|---|---|---|
| Bst DNA polymerase | 320 | U/mL | 1 |

| | | |
|---|---|---|
| LAMP dye | 1× | 0.5 |

| | | | |
|---|---|---|---|
| F3 | 200 | nM | 1 |
| B3 | 200 | nM | 1 |
| FIP | 1600 | nM | 1 |
| BIP | 1600 | nM | 1 |
| LF | 400 | nM | 0.25 |
| LB | 400 | nM | 0.25 |

| | | |
|---|---|---|
| Standard substance | / | 1 |
| TRIS EDTA (TE) 0.1 | / | 5.5 |

| | |
|---|---|
| Total system | 25 |

### (3) Reaction Program:

The reaction is maintained at a constant temperature of 65° C. for 60 min. In the prior art, base primers and loop primers are designed based on optimization of Tm value, dG value, GC content, and distance between primers using LAMP and PrimerExplorer V5 software. The standard substance, amplification primers, amplification reagents, and water are configured into a 25 μL LAMP reaction system to allow isothermal amplification at 65° C., so as to obtain an exponential amplification inflection point time (min) within 40 min.

The PCR instrument used in all the following examples is a Roche LightCycler 48011 fluorescence quantitative PCR instrument.

### Example 1 Design and Optimization of Base Primers

In the present disclosure, a primer of the LAMP system was designed for a specific sequence region of a DNA sequence of a pMD18-T plasmid. The base primers (F3, B3, FIP, BIP) were designed using PrimerExplorer V5 software, and the base primers were obtained by optimizing the Tm value, dG value, GC content, and distance between primers.

The specific sequence was as follows:

SEQ ID NO: 13:
ACTCTTCCAG CCTTCCTTCC TGGGTGAGTG GAGACTGTAAT
CTTGGCTCAC CCTCATGAGGGTTACCCCTCGGGGCTGTGCTGTGGAAGC
TATCTCCTGCCCTCATTTCCCTCTCAGGCATGGAGTCCTGTGGCATCCAC
GTTTCTACCTTCAACTCCATCATGAAGTGTCTGGTGGACATCCGCAAAGA
CCTGTACGCAACACAGTGCTGTCTCCGCGCACCACCATCCAAGGGGTGGC
ATTGCCGACAGGATGCAGAA GGAGATCACT GCCCTGGCAC
CCAGCACAAT CTTGATCTTG.

A DNA plasmid was used as a template, and its copy number concentration was diluted to 10² copies/μL, and a reaction system was configured according to the composition in Table 4:

TABLE 4

| Component | Working concentration | | Volume (μL) |
|---|---|---|---|
| Reaction buffer | 1× | | 12.5 |
| Bst DNA polymerase | 320 | U/mL | 1 |
| LAMP dye | 1× | | 0.5 |
| F3 | 200 | nM | 1 |
| B3 | 200 | nM | 1 |
| FIP | 1600 | nM | 1 |
| BIP | 1600 | nM | 1 |

| | | |
|---|---|---|
| Standard substance | / | 1 |
| TE 0.1 (TE buffer, including | / | 6 |
| EDTA 0.1 mM and Tris 10 mM | | |
| by concentration) | | |
| Total system | | 25 |

A temperature cycle process of the LAMP reaction was shown in Table 5:

TABLE 5

| | Temperature (° C.) | Time | Number of cycles |
|---|---|---|---|
| Isothermal amplification | 65 | 1 min | 60 |

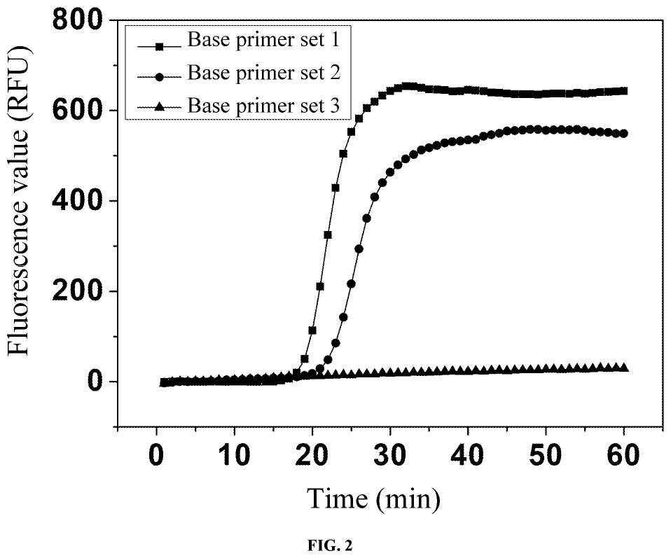

Readings during LAMP amplification were obtained in real time based on the Roche LightCycler 48011 fluorescence quantitative PCR instrument. In the reaction system of the base primer set, there was obvious amplification of template DNA. The exponential amplification inflection point time (min) automatically read by the instrument is shown in Table 6 to Table 9 while the amplification results are shown in FIG. 2:

TABLE 6

| | Exponential amplification |
|---|---|
| | inflection point time (min) |
| Base primer set 1 | 19.65 |

The experiment was repeated with the base primer set, and the amplification effect is shown in FIG. 2, with desirable repeatability.

TABLE 7
Sequences of base primer set 1

| Primer name | Sequence |
|---|---|
| F3 (SEQ ID NO: 1) | CTGGGTGAAAGGAGACTGT |
| B3 (SEQ ID NO: 2) | AGACAGGGCTGTGTTGGC |
| FIP (SEQ ID NO: 3) | TGAGAGGGAAATGATTTCAGGACATGAGGGTTACCCCTCGG |
| BIP (SEQ ID NO: 4) | GGCATGGAGTCCTCACCCATCGTACAGGTCTTTGCGGATGT |

TABLE 8
Base primer set 2
(exponential amplification inflection point time (min): 21.98)

| Primer name | Sequence |
|---|---|
| F3 (SEQ ID NO: 5) | ATGAGGGTTACCCCTCGG |
| B3 (SEQ ID NO: 6) | TGCATCCTGTCGGCAATG |
| FIP (SEQ ID NO: 7) | GCCACAGGACTCCATGCCTGGCTGTGCTGTGGAAGCTAAG |
| BIP (SEQ ID NO: 8) | TGAAGTGTGACGTGGACATCCGCAGGGTACATGGTGGTGC |

TABLE 9
Base primer set 3 (no amplification achieved)

| Primer name | Sequence |
|---|---|
| F3 (SEQ ID NO: 9) | ACTCTTCCAGCCTTCCTTCC |
| B3 (SEQ ID NO: 10) | GCGTACAGGTCTTTGCGG |
| FIP (SEQ ID NO: 11) | TAGCTTCCACAGCACAGCCCTGGGTGAGTGGAGACTGTC |
| BIP (SEQ ID NO: 12) | TTCCCTCTCAGGCATGGAGTCCTGTCCACGTCACACTTCATG |

### Example 2 Design and Optimization of Loop Primers

In the present disclosure, a primer of the LAMP system was designed for a specific sequence region of a DNA sequence of a plasmid. The base primers (F3, B3, FIP, BIP) were designed using PrimerExplorer V5 software, and the loop primer set was obtained by optimizing the Tm value, dG value, GC content, and distance between primers.

pMD18-T plasmid; the specific sequence was as follows:

SEQ ID NO: 13:
ACTCTTCCAG CCTTCCTTCC TGGGTGAGTG GAGACTGTAAT
CTTGGCTCAC CCTCATGAGGGTTACCCCTCGGGGCTGTGCTGTGGAAGC
TATCTCCTGCCCTCATTTCCCTCTCAGGCATGGAGTCCTGTGGCATCCAC
GTTTCTACCTTCAACTCCATCATGAAGTGTCTGGTGGACATCCGCAAAGA
CCTGTACGCAACACAGTGCTGTCTCCGCGCACCACCATCCAAGGGGTGGC
ATTGCCGACAGGATGCAGAA GGAGATCACT GCCCTGGCAC
CCAGCACAAT CTTGATCTTG.

A DNA plasmid was used as a template, and its copy number concentration was diluted to 10² copies/μL, and a reaction system was configured according to the composition in Table 10:

TABLE 10

| | | |
|---|---|---|
| Component | Working concentration | Volume (μL) |

| | | |
|---|---|---|
| Reaction buffer | 1× | 12.5 |

| | | | |
|---|---|---|---|
| Bst DNA polymerase | 320 | U/mL | 1 |

| | | |
|---|---|---|
| LAMP dye | 1× | 0.5 |

| | | | |
|---|---|---|---|
| F3 | 200 | nM | 1 |
| B3 | 200 | nM | 1 |
| FIP | 1600 | nM | 1 |
| BIP | 1600 | nM | 1 |
| LF | 400 | nM | 0.25 |
| LB | 400 | nM | 0.25 |

| | | |
|---|---|---|
| Standard substance | / | 1 |
| TE 0.1 | / | 5.5 |
| Total system | | 25 |

A temperature cycle process of the LAMP reaction was shown in Table 11:

TABLE 11

| | Temperature (° C.) | Time | Number of cycles |
|---|---|---|---|
| Isothermal amplification | 65 | 1 min | 60 |

Readings were obtained after LAMP amplification was completed. In all reaction systems containing base primers and loop primers, there was obvious amplification of template DNA, and the exponential amplification inflection point time (min) automatically read by the instrument was shown in Table 12 to Table 15:

TABLE 12

| | Exponential amplification |
|---|---|
| | inflection point time (min) |
| LAMP 1-1 primer set (including | 12.26 |
| base primers and loop primers) | |

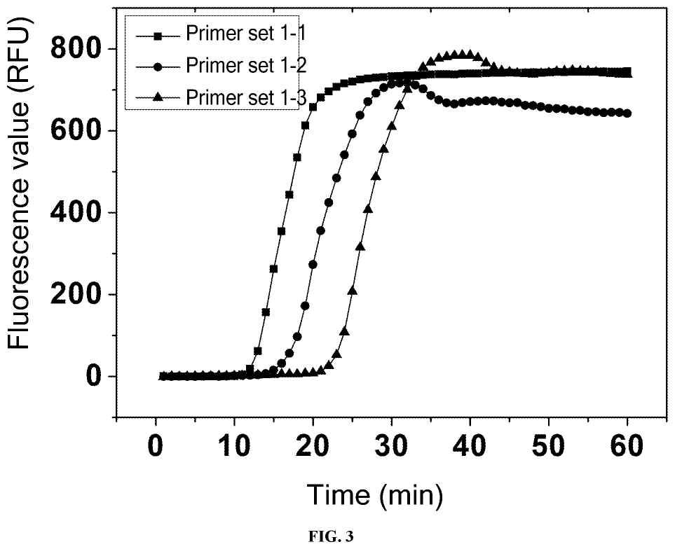

The experiment was repeated, and the amplification effect was shown in FIG. 3, with desirable repeatability.

TABLE 13
Sequences of LAMP 1-1 primer set

| Primer name | Sequence |
|---|---|
| F3 (SEQ ID NO: 1) | CTGGGTGAAAGGAGACTGT |
| B3 (SEQ ID NO: 2) | AGACAGGGCTGTGTTGGC |
| FIP (SEQ ID NO: 3) | TGAGAGGGAAATGATTTCAGGACATGAGGGTTACCCCTCGG |
| BIP (SEQ ID NO: 4) | GGCATGGAGTCCTCACCCATCGTACAGGTCTTTGCGGATGT |
| LF (SEQ ID NO: 14) | TAGCTTAATCAGGAGAGCC |
| LB (SEQ ID NO: 15) | ACCTTCTTCTCCATCATGAAGTG |

TABLE 14
1-2 primer set
(exponential amplification inflection point time (min): 16.87)

| Primer name | Sequence |
|---|---|
| F3 (SEQ ID NO: 1) | CTGGGTGAAAGGAGACTGT |
| B3 (SEQ ID NO: 2) | AGACAGGGCTGTGTTGGC |
| FIP (SEQ ID NO: 3) | TGAGAGGGAAATGATTTCAGGACATGAGGGTTACCCCTCGG |
| BIP (SEQ ID NO: 4) | GGCATGGAGTCCTCACCCATCGTACAGGTCTTTGCGGATGT |
| LF (SEQ ID NO: 16) | TTAGCTTCCACAGCACAGCC |
| LB (SEQ ID NO: 17) | TTCAACTCCATCATGAAGTGTGAC |

TABLE 15
1-3 primer set
(exponential amplification inflection point time (min): 22.75)

| Primer name | Sequence |
|---|---|
| F3 (SEQ ID NO: 1) | CTGGGTGAAAGGAGACTGT |
| B3 (SEQ ID NO: 2) | AGACAGGGCTGTGTTGGC |
| FIP (SEQ ID NO: 3) | TGAGAGGGAAATGATTTCAGGACATGAGGGTTACCCCTCGG |
| BIP (SEQ ID NO: 4) | GGCATGGAGTCCTCACCCATCGTACAGGTCTTTGCGGATGT |
| LB (SEQ ID NO: 18) | CTCCATCATGAAGTGTGACGTG |

### Example 3 Optimization of Amplification Temperature

In this example, the amplification temperature of the LAMP reaction was optimized, and an amplification temperature gradient was set to: 64° C., 65° C., and 66° C.

A DNA plasmid was used as a template, and its copy number concentration was diluted to 10² copies/μL, and a reaction system was configured using primers shown in SEQ ID NO: 1 to SEQ ID NO: 6 according to the composition in Table 16:

TABLE 16

| | | |
|---|---|---|
| Component | Working concentration | Volume (μL) |

| | | |
|---|---|---|
| Reaction buffer | 1× | 12.5 |

| | | | |
|---|---|---|---|
| Bst DNA polymerase | 320 | U/mL | 1 |

| | | |
|---|---|---|
| LAMP dye | 1× | 0.5 |

| | | | |
|---|---|---|---|
| F3 | 200 | nM | 1 |
| B3 | 200 | nM | 1 |
| FIP | 1600 | nM | 1 |
| BIP | 1600 | nM | 1 |
| LF | 400 | nM | 0.25 |
| LB | 400 | nM | 0.25 |

| | | |
|---|---|---|
| Standard substance | / | 1 |
| TE 0.1 | / | 5.5 |
| Total system | | 25 |

The temperature cycle process of the LAMP reaction is shown in Table 17:

TABLE 17

| | Temperature (° C.) | Time | Number of cycles |
|---|---|---|---|
| Isothermal amplification | 64/65/66 | 1 min | 60 |

Readings were obtained after LAMP amplification was completed. In reaction systems of 64° C./65° C./66° C., there was obvious amplification of template DNA, and the exponential amplification inflection point time (min) automatically read by the instrument is shown in Table 18:

TABLE 18

| | Exponential amplification |
|---|---|
| Temperature (° C.) | inflection point time (min) |
| 64 | 16.29 |
| 65 | 12.26 |
| 66 | 14.28 |

Based on this, the amplification temperature of the reaction system was finally determined to be 65° C.

### Example 4 Determination for Measurement Range of LAMP Reaction

A DNA plasmid was used as a template, and its copy number concentration was gradient diluted to (10⁰-10⁶) copies/μL, and a reaction system was configured using primers shown in SEQ ID NO: 1 to SEQ TD NO: 6 according to the composition in Table 19:

TABLE 19

| | | |
|---|---|---|
| Component | Working concentration | Volume (μL) |

| | | |
|---|---|---|
| Reaction buffer | 1× | 12.5 |

| | | | |
|---|---|---|---|
| Bst DNA polymerase | 320 | U/mL | 1 |

| | | |
|---|---|---|
| LAMP dye | 1× | 0.5 |

| | | | |
|---|---|---|---|
| F3 | 200 | nM | 1 |
| B3 | 200 | nM | 1 |
| FIP | 1600 | nM | 1 |
| BIP | 1600 | nM | 1 |
| LF | 400 | nM | 0.25 |
| LB | 400 | nM | 0.25 |

| | | |
|---|---|---|
| Standard substance | / | 1 |
| TE 0.1 | / | 5.5 |
| Total system | | 25 |

The temperature cycle process of the LAMP reaction is shown in Table 20:

TABLE 20

| | Temperature (° C.) | Time | Number of cycles |
|---|---|---|---|
| Isothermal amplification | 65 | 1 min | 60 |

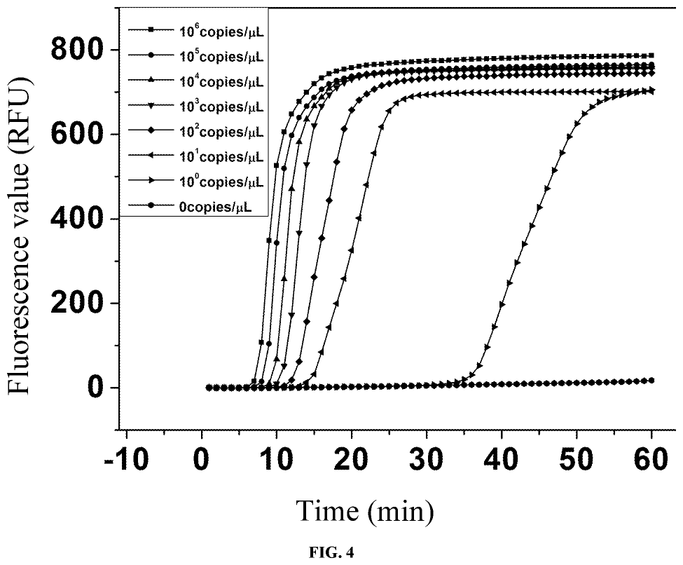

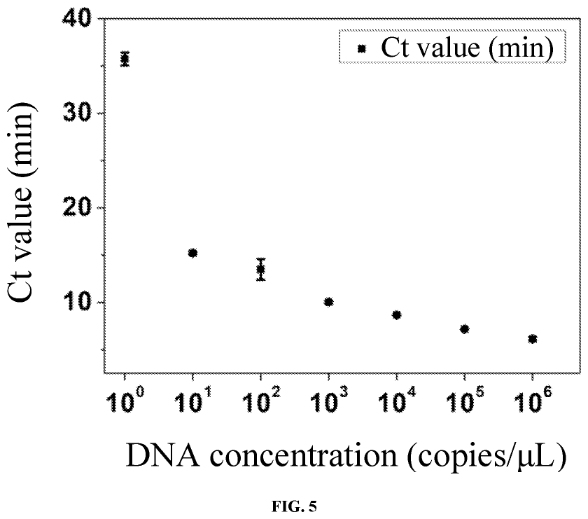

LAMP was completed and readings were obtained, and the amplification effect is shown in FIG. 4. After repeated experiments, an average exponential amplification inflection point time (min) (FIG. 5) is shown in Table 21:

TABLE 21

| | Exponential amplification inflection | | | | |
|---|---|---|---|---|---|
| DNA | point time (min) | | | | |
| concentration | | | | Average | |
| (copies/μL) | 1 | 2 | 3 | value | RSD |
| 10⁶ | 6.00 | 6.36 | 6.00 | 6.12 | 3.40% |
| 10⁵ | 7.21 | 7.17 | 7.11 | 7.16 | 0.70% |
| 10⁴ | 8.63 | 8.66 | 8.7 | 8.66 | 0.41% |
| 10³ | 10.03 | 10.14 | 9.88 | 10.02 | 1.30% |
| 10² | 12.26 | 14.36 | 13.82 | 13.48 | 8.09% |
| 10¹ | 15.06 | 15.28 | 15.28 | 15.21 | 0.84% |
| 10⁰ | 36.51 | 35.34 | 35.34 | 35.73 | 1.89% |

Therefore, the reaction measurement of this kit for calibration of an isothermal PCR instrument ranged from (10⁰-10⁶) copies/μL.

### Example 5 Example for Calibration of Isothermal Amplification PCR Instrument

The isothermal PCR instrument was calibrated based on the kit and method of the present disclosure, and a calibration system was a reaction system configured according to the following Table 22.

TABLE 22

| | | |
|---|---|---|
| Component | Working concentration | Volume (μL) |

| | | |
|---|---|---|
| Reaction buffer | 1× | 12.5 |

| | | | |
|---|---|---|---|
| Bst DNA polymerase | 320 | U/mL | 1 |

| | | |
|---|---|---|
| LAMP dye | 1× | 0.5 |

| | | | |
|---|---|---|---|
| F3 | 200 | nM | 1 |
| B3 | 200 | nM | 1 |
| FIP | 1600 | nM | 1 |
| BIP | 1600 | nM | 1 |
| LF | 400 | nM | 0.25 |
| LB | 400 | nM | 0.25 |

| | | |
|---|---|---|
| Standard substance | / | 1 |
| TE 0.1 | / | 5.5 |
| Total system | | 25 |

The temperature cycle process of the LAMP reaction is shown in Table 23:

TABLE 23

| | Temperature (° C.) | Time | Number of cycles |
|---|---|---|---|
| Isothermal amplification | 65 | 1 min | 60 |

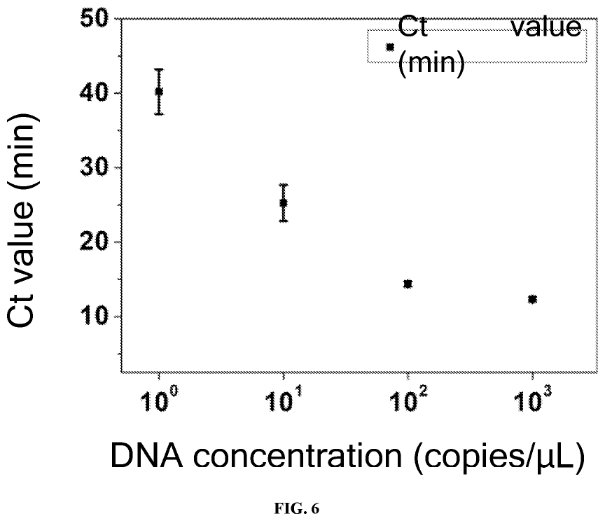

Readings were obtained after the LAMP amplification was completed. After repeated experiments, an average exponential amplification inflection point time (min) (FIG. 6) is shown in Table 24.

TABLE 24

| | Exponential amplification |
|---|---|
| DNA concentration (copies/μL) | inflection point time (min) |
| 10³ | 12.32 |
| 10² | 14.38 |
| 10¹ | 25.26 |
| 10⁰ | 40.20 |

### Calibration Results:

(1) sensitivity: 10⁰ copies/μL; (2) measurement range: 10⁰ copies/μL to 103 copies/μL; (3) measurement repeatability.

The above examples are only intended to describe the preferred implementations of the present disclosure, but not to limit the scope of the present disclosure. Various alterations and improvements made by those of ordinary skill in the art based on the technical solution of the present disclosure without departing from the design spirit of the present disclosure shall fall within the scope of the appended claims of the present disclosure.

What is claimed is:

1. A primer set for calibration of an isothermal polymerase chain reaction (PCR) analyzer, wherein the primer set comprises the nucleotide sequences shown in SEQ ID NO: 1 to SEQ ID NO: 6.

2. A kit for calibration of an isothermal PCR analyzer, comprising the primer set according to claim 1.

3. The kit according to claim 2, further comprising a standard substance, a reaction buffer, a polymerase, and a dye.

4. The kit according to claim 3, wherein the standard substance is a DNA plasmid with a gradient concentration of 10⁰ copies/μL to 10⁶ copies/μL.

5. The kit according to claim 4, wherein the DNA plasmid is a pMD18 plasmid with a specific sequence shown in SEQ ID NO: 13.

6. The kit according to claim 5, wherein the reaction buffer comprises the following components: 20 mM of Tris-HCl, 10 mM of (NH₄)₂SO₄, 50 mM of KCl, 8 mM of MgSO₄, 0.1% of Tween 20, and 1.4 mM each of dNTPs; and the polymerase is a Bst DNA polymerase.

7. The kit according to claim 6, further comprising a negative control.

8. A loop-mediated isothermal amplification (LAMP) reaction system for calibration of an isothermal PCR instrument, wherein the LAMP reaction system has a volume of 25 μL and comprises the following components: 12.5 μL of 2× reaction buffer, 1 μL of Bst DNA polymerase, 0.5 μL of 50×LAMP dye, 5 pmol of SEQ ID NO: 1, 5 pmol of SEQ ID NO: 2, 40 pmol of SEQ ID NO: 3, 40 pmol of SEQ ID NO: 4. 10 pmol of SEQ ID NO: 5, 10 pmol of SEQ ID NO: 6, and 1 μL of the standard substance according to claim 5, and ultrapure water added to make up to 25 μL.

9. A method for calibrating an isothermal PCR analyzer, comprising: designing the primer set according to claim 1 and applying the primer set in the process of calibration of the isothermal PCR analyzer.

10. A method for calibrating an isothermal PCR analyzer, comprising applying the kit according to claim 2 in the process of calibration of the isothermal PCR analyzer.

11. The method according to claim 10, wherein the kit further comprises a standard substance, a reaction buffer, a polymerase, and a dye.

12. The method according to claim 11, wherein the standard substance is a DNA plasmid with a gradient concentration of 10⁰ copies/μL to 10⁶ copies/μL.

13. The method according to claim 12, wherein the DNA plasmid is a pMD18 plasmid with a specific sequence shown in SEQ ID NO: 13.

14. The method according to claim 13, wherein the reaction buffer comprises the following components: 20 mM of Tris-HCl, 10 mM of (NH₄)₂SO₄, 50 mM of KCl, 8 mM of MgSO₄, 0.1% of Tween 20, and 1.4 mM each of dNTPs; and the polymerase is a Bst DNA polymerase.

15. The method according to claim 14, wherein the kit further comprises a negative control.


SEQUENCE LISTING (18 sequences)

SEQ ID NO: 1 - DNA, 19 bp, synthetic construct
  source 1..19: mol_type = other DNA; note = Nucleotide sequence of primer F3 for PCR amplication
ctgggtgaaaggagactgt

SEQ ID NO: 2 - DNA, 18 bp, synthetic construct
  source 1..18: mol_type = other DNA; note = Nucleotide sequence of primer B3 for PCR amplication
agacagggctgtgttggc

SEQ ID NO: 3 - DNA, 41 bp, synthetic construct
  source 1..41: mol_type = other DNA; note = Nucleotide sequence of primer FIP for PCR amplication
tgagagggaaatgatttcaggacatgagggttacccctcgg

SEQ ID NO: 4 - DNA, 41 bp, synthetic construct
  source 1..41: mol_type = other DNA; note = Nucleotide sequence of primer BIP for PCR amplication
ggcatggagtcctcacccatcgtacaggtctttgcggatgt

SEQ ID NO: 5 - DNA, 19 bp, synthetic construct
  source 1..19: mol_type = other DNA; note = Nucleotide sequence of primer LF for PCR amplication
tagcttaatcaggagagcc

SEQ ID NO: 6 - DNA, 23 bp, synthetic construct
  source 1..23: mol_type = other DNA; note = Nucleotide sequence of primer LB for PCR amplication
accttcttctccatcatgaagtg

SEQ ID NO: 7 - DNA, 40 bp, synthetic construct
  source 1..40: mol_type = other DNA; note = Nucleotide sequence of base primer FIP for loop-mediated isothermal amplification
gccacaggactccatgcctggctgtgctgtggaagctaag

SEQ ID NO: 8 - DNA, 40 bp, synthetic construct
  source 1..40: mol_type = other DNA; note = Nucleotide sequence of base primer BIP for loop-mediated isothermal amplification
tgaagtgtgacgtggacatccgcagggtacatggtggtgc

SEQ ID NO: 9 - DNA, 20 bp, synthetic construct
  source 1..20: mol_type = other DNA; note = Nucleotide sequence of base primer F3 for loop-mediated isothermal amplification (no amplification achieved)
actcttccagccttccttcc

SEQ ID NO: 10 - DNA, 18 bp, synthetic construct
  source 1..18: mol_type = other DNA; note = Nucleotide sequence of base primer B3 for loop-mediated isothermal amplification (no amplification achieved)
gcgtacaggtctttgcgg

SEQ ID NO: 11 - DNA, 39 bp, synthetic construct
  source 1..39: mol_type = other DNA; note = Nucleotide sequence of base primer FIP for loop-mediated isothermal amplification (no amplification achieved)
tagcttccacagcacagccctgggtgagtggagactgtc

SEQ ID NO: 12 - DNA, 42 bp, synthetic construct
  source 1..42: mol_type = other DNA; note = Nucleotide sequence of base primer BIP for loop-mediated isothermal amplification (no amplification achieved)
ttccctctcaggcatggagtcctgtccacgtcacacttcatg

SEQ ID NO: 13 - DNA, 300 bp, synthetic construct
  source 1..300: mol_type = other DNA; note = Sequence of plasmid pMD18-T
actcttccagccttccttcctgggtgagtggagactgtaatcttggctcaccctcatgagggttacccctcggggctgtgctgtggaagctatctcctgccctcatttccctctcaggcatggagtcctgtggcatccacgtttctaccttcaactccatcatgaagtgtctggtggacatccgcaaagacctgtacgcaacacagtgctgtctccgcgcaccaccatccaaggggtggcattgccgacaggatgcagaaggagatcactgccctggcacccagcacaatcttgatcttg

SEQ ID NO: 14 - DNA, 19 bp, synthetic construct
  source 1..19: mol_type = other DNA; note = Nucleotide sequence of base primer LF for loop-mediated isothermal amplification
tagcttaatcaggagagcc

SEQ ID NO: 15 - DNA, 23 bp, synthetic construct
  source 1..23: mol_type = other DNA; note = Nucleotide sequence of base primer LB for loop-mediated isothermal amplification
accttcttctccatcatgaagtg

SEQ ID NO: 16 - DNA, 20 bp, synthetic construct
  source 1..20: mol_type = other DNA; note = Nucleotide sequence of base primer LF for loop-mediated isothermal amplification (showing exponential amplification at exponential amplification at inflection point time 16.87min)
ttagcttccacagcacagcc

SEQ ID NO: 17 - DNA, 24 bp, synthetic construct
  source 1..24: mol_type = other DNA; note = Nucleotide sequence of base primer LB for loop-mediated isothermal amplification (showing exponential amplification at inflection point time 16.87min)
ttcaactccatcatgaagtgtgac

SEQ ID NO: 18 - DNA, 22 bp, synthetic construct
  source 1..22: mol_type = other DNA; note = Nucleotide sequence of base primer LB for loop-mediated isothermal amplification (showing exponential amplification)
ctccatcatgaagtgtgacgtg